(19) United States
(12) Patent Application Publication
(10) Pub. No.: US 2007/0135871 A1
(43) Pub. Date: Jun. 14, 2007

(54) Use of stellate ganglion block for the treatment of psychiatric and behavioral disorders

(76) Inventor: Eugene Lipov, Chicago, IL (US)

Correspondence Address: BRIAN J. LUM;ICE MILLER LLP, SUITE 3500, 200 WEST MADISON STREET, CHICAGO, IL, 60606, US

(21) Appl. No.: 11/702,297

(22) Filed: Feb. 5, 2007

Related U.S. Application Data
(63) Continuation-in-part of application No. 11/411,468, filed on Apr. 25, 2006.
(60) Provisional application No. 60/691,055, filed on Jun. 16, 2005.
(60) Provisional application No. 60/697,233, filed on Jul. 7, 2005.

(51) Int. Cl. A61N 5/06

(52) U.S. Cl. 607/89

(57) ABSTRACT

The present invention is directed to a method for the treatment of a patient suffering from psychiatric and behavioral disorders, including post partum depression, post traumatic stress disorder, compulsive smoking, attention deficit hyperactivity disorder, gambling addiction, comprising the step of administering a stellate ganglion block to the patient to alleviate the symptoms. The stellate ganglion block may be followed by a sympathectomy to provide permanent relief.

This application is a continuation-in-part of U.S. application Ser. No. 11/411,468, filed Apr. 25, 2006, and claims priority to U.S. Provisional Application No. 60/691,055, filed Jun. 16, 2005, and U.S. Provisional Application No. 60/697,233, filed Jul. 7, 2005, herein incorporated by reference.

### BACKGROUND OF THE INVENTION

The present invention is directed to methods and kits for the treatment of hot flashes other symptoms of menopause, and other conditions.

Hot flashes are experienced by many women undergoing menopause. A hot flash is a feeling of warmth, sometimes associated with flushing, that spreads over the body and may be accompanied by perspiration. Hot flashes may vary in severity and may last for a short period of time in some women, but can last for a decade or more in other women. Although the cause of hot flashes is not completely understood, hot flashes may be related to fluctuations of hormone levels experienced during menopause. Hot flashes also may be experienced secondary to mastectomy and other cancer-related treatments, particularly cancer treatments that affect hormone levels.

Traditionally, hot flashes have been treated with hormone therapy. Hormone replacement medications (usually estrogen or a combination of estrogen and progesterone) are effective in reducing the frequency of hot flashes and their severity. Generally, these medications decrease the frequency of hot flashes by about 80 to 90%. However, hormone replacement therapy is associated with increased risk of heart attack, stroke, blood clots, and breast cancer. Thus, alternative treatments are desirable.

A stellate ganglion block is an injection of local anesthetic into the stellate ganglion nerves—nerve tissue located in the neck on either side of the larynx. The injection of local anesthetic into these nerves blocks impulses, which may in turn reduce pain, swelling, color, and sweating changes in the upper extremity and may improve mobility. Stellate ganglion blocks are known as part of the treatment of Reflex Sympathetic Dystrophy (RSD), Sympathetic Maintained Pain, Complex Regional Pain Syndrome (CRPS), and Herpes Zoster (shingles) involving the head and face and/or upper extremities.

It has been found that the stellate ganglion block can be used to treat hot flashes. In an initial study, complete asymptomatic relief of two to five weeks has been experienced with a single stellate ganglion block, with increasingly longer periods of relief following subsequent blocks.

### SUMMARY OF THE INVENTION

The present invention is directed to a method for the treatment of a patient suffering from a menopause-related condition comprising the step of administering a stellate ganglion block to provide relief from the menopause-related condition. Illustratively, the patient is suffering from hot flashes and the stellate ganglion block alleviates the symptoms of hot flashes.

Additional features of the present invention will become apparent to those skilled in the art upon consideration of the following detailed description of preferred embodiments exemplifying the best mode of carrying out the invention as presently perceived.

### BRIEF DESCRIPTION OF THE DRAWINGS

FIG. 1 is a perspective view showing various vertebrae and nerves in the neck and needle placement for a stellate ganglion block.

### DETAILED DESCRIPTION

A ganglion is a group of nerve cell bodies. The stellate ganglion is a ganglion located in the neck, formed by the fusion of the inferior cervical and the first thoracic ganglions. These ganglions meet anterior to the C7 vertebra.

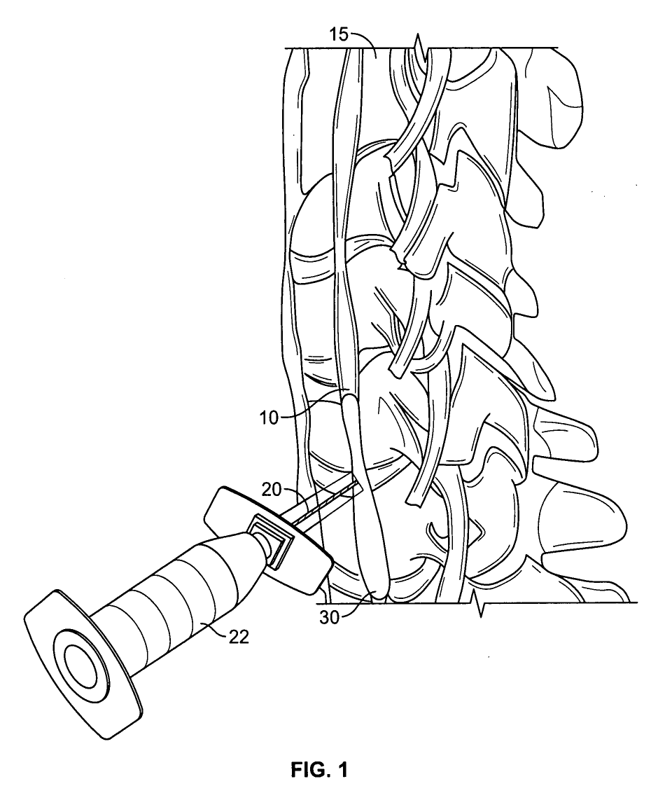

A stellate ganglion block is illustratively performed by placing the patient in the supine position (on the back) with the neck slightly extended and the jaw open. While the patient is usually conscious for the procedure, a medication such as Versed (midazolam hydrochloride), fentanyl, or other sedative is often administered, to relax the patient. A local anesthetic is also usually administered, to numb the skin and tissue down to the ganglion nerve. A needle is inserted toward the stellate ganglion. FIG. 1 shows the stellate ganglion 10 extending anterior to vertebral column 15. The needle 20 is shown inserted adjacent to the stellate ganglion 10. In the illustrative block, a contrast solution is injected through needle 20 via syringe 22, and the needle position is confirmed by fluoroscopy or other means, as are known in the art.

The anesthetic 30 is then delivered into stellate ganglion 10 by injection via needle 20. Illustratively, 5 cc 0.375% Marcaine® (bupivacaine) may be used. However, it is understood that other anesthetics, illustratively lidocaine, morphine or other opioids, clonidine, botox, or tetracine, or a combination of anesthetic and epinephrine, may be used and that the dosage range may vary depending on the particular anesthetic and other factors, as are known in the art. Needle 20 is removed, and the patient is optionally placed in the sitting position to facilitate the spread of anesthesia inferiorly to the stellate ganglion. Illustratively, longer acting anesthetics such as bupivacaine are used, but other anesthetics such as narcan may be used, as are known in the art. The stellate ganglion block may also be performed by radiofrequency lesioning of the stellate ganglion or linear polarized light irradiation near the stellate ganglion, as is known in the art.

While fluoroscopy is used in the illustrative method, stellate ganglion blocks have been traditionally performed blindly by palpating the transverse process of C6 and infiltrating a large volume (as much as 20 mL) of local anesthetic. It is understood that this technique is dependent on enough volume reaching the stellate ganglion to result in an effective block and requires a larger volume of anesthetic. While such techniques may be used to treat hot flashes, it is understood that image-guided stellate ganglion blocks have the advantages of increased safety and accuracy compared with blind injections because the needle can be accurately placed close to the ganglion itself. Because of the more accurate placement of the needle to the ganglion, a smaller amount of local anesthetic can be used, resulting in a decrease in adverse effects.

Patients may experience side effects such as nasal congestion, a hoarse voice, and a warm tingling sensation in the arm and hand, and may display symptoms associated with “Horner's syndrome” such as a bloodshot, droopy eye on the side of the injection. These side effects usually disappear after several hours. If the first injection reduces or eliminates hot flashes for a period of time, subsequent blocks may be given. It has been found that the duration of relief often increases with each subsequent injection. Surgical sympathectomy may be performed for permanent relief from hot flashes. Sympathectomies are currently performed on patients who suffer from excessive perspiration and the surgical techniques are known in the art. Generally, a sympathectomy would be performed if a patient responds well to the stellate ganglion block, to provide permanent relief from severe hot flashes.

In addition to hot flashes, it is believed that stellate ganglion blocks may aid in the treatment and/or prevention of other conditions associated with menopause, including osteoporosis, fat deposits and sexual dysfunction. For ongoing medical conditions such as osteoporosis, a series of stellate ganglion blocks may be administered periodically, for months or years. The blocks may be administered every week or two to every several months. The blocks may be administered on a regular periodic basis, or the length of time between consecutive blocks may change, illustratively increasing over time. The stellate ganglion block may also prove to be an effective treatment for overweight patients, resulting in weight loss and/or prevention or treatment of diabetes mellitus. A series of additional stellate ganglion blocks may be administered periodically, until a desired weight is attained or to maintain a desired weight. Finally, the stellate ganglion block may be effective for mood disorders, schizophrenia, and other psychiatric disorders, with repeat treatments as necessary. A sympathectomy may also be effective for these conditions.

The stellate ganglion block is believed to be particularly effective in treating post partum depression, a mood disorder experienced by some women following child birth. Most women experience the “baby blues” after delivery, becoming emotionally sensitive, tearful and/or moody. These symptoms are typically resolved without treatment within 1 or 2 weeks. However, in cases of post partum depression, these feelings may persist for weeks or months after child birth and may include difficulty sleeping, fatigue, feelings of inadequacy and/or impaired concentration. In rare cases, these symptoms are severe and the subject may become suicidal, may consider killing her child and/or may become psychotic, experiencing delusions or hallucinations.

Post partum depression is believed to be linked to the fluctuations in hormone levels that occur after child birth, including the sharp drop in estrogen levels. In many cases, post partum depression has been successfully treated using estrogen delivered transdermally via skin patches. Increased estrogen levels are known to modulate the activity of the amygdala, a region of the brain that is believed to participate in processing of emotions in response to external stimuli, such as fear and anxiety. Increased or abnormal activity in the amygdala has been implicated in depression. Thus, it is possible that the use of estrogen therapy to treat post partum depression operates by reducing activity in the amygdala.

Signals from the amygdala are output to the sympathetic branch of the autonomic nervous system, of which the stellate ganglion is a part, and evidence suggests that the amygdala and sympathetic system are directly connected. Furthermore, depression is typically characterized by increased activity of the sympathetic system and estrogen is found to reduce sympathetic activity. Without wishing to be limited to any particular theory, the use of the stellate ganglion block to treat post partum depression may operate by modulating or disrupting the ability of the amygdala to control the sympathetic system and cause depression.

Treatment of post partum depression using the stellate ganglion block may be performed in the same manner as described above for treatment of hot flashes—e.g., by injection of anesthetics such as bupivacaine into the stellate ganglion. Subsequent stellate ganglion blocks may be required in cases where post partum depression persists longer than a few weeks. In extreme cases of post partum depression, radiofrequency lesioning or surgical sympathectomy may be required.

In addition to post partum depression, stellate ganglion block is also believed to be useful in the treatment of other psychiatric and behavioral disorders. In particular, post traumatic stress disorder (PTSD) is an anxiety disorder that may follow a traumatic experience, such as a life threatening event. PTSD is characterized by repeated flashbacks, nightmares and other recurrent memories of the traumatic event persisting for a period of more than a month, and is frequently accompanied by sleep disturbances, emotional numbness, depression, difficulty concentrating, exaggerated startle response, hyper vigilance, anxiety, irritability, outbursts of anger and/or other symptoms known in the art. As discussed above, such symptoms may implicate the amygdala. Persons suffering from PTSD may also experience physical symptoms, such as headaches, stomachaches, dizziness, chest pain, difficulty breathing, discomfort and/or immune systems problems. In some cases, PTSD has been found to associated with increased activity of the sympathetic nervous system, which may account for some of the observed physical and psychological symptoms.

The autonomic nervous system is also believed to play a role in addictive behavior, cravings, habits and automatic or implicit urges, such as compulsive smoking and nicotine addiction. It has been shown that in some cases, cigarette smokers who suffer from damage to the insular cortex (also referred to as the “insula”) lose the urge to smoke. The insula is implicated in the regulation of the autonomic nervous system. Stimulation of the right insular cortex has been shown to increase activity of the sympathetic nervous system, whereas stimulation of the left insular cortex has parasympathetic effect. Furthermore, the Furthermore, the insula is connected to and receives input from the amygdale. Thus, the insula may serve as a gateway for the sympathetic/parasympathetic nervous system.

Stellate ganglion block may be used to treat disorders such as PTSD and to provide relief from the urge to smoke in the same manner as described above for post partum depression. In a preferred embodiment, the stellate ganglion block is performed by C-6 injection of anesthetic, but may also be administered by other means, such as radiofrequency lesioning, linear polarized light irradiation or surgical sympathectomy, as discussed above. Repeated treatment may be used where the problem persists.

Other psychiatric and behavioral disorders are believed to be similarly responsive to treatment by stellate ganglion block, including attention deficit hyperactivity disorder (ADHD) and addiction disorders, such as pathological gambling. ADHD appears early in a child's life and is characterized by inattention, hyperactivity and impulsivity. Other behavioral disorders are commonly associated with ADHD, including oppositional defiant disorder and conduct disorder characterized by stubbornness, belligerence and more serious antisocial behavior. Treatment for ADHD is presently limited to behavioral therapy and/or medication such as stimulants. Neurological studies suggest reduced activity of the orbitofrontal cortex and anterior cingulate cortex in subjects with ADHD.

Pathological gambling is characterized by a chronic and progressive preoccupation with gambling, which leads to behavior that disrupts relationships and daily activities. Treatment is presently limited to behavioral therapy and pharmacotherapy for any coexisting depression. Studies on the neurological basis of pathological gambling have implicated impairment of decision making associated with orbitofrontal cortex dysfunction and anterior cingulated cortex.

Both the orbitofrontal cortex and anterior cingulate cortex are believed to be linked to the insular cortex and amygdala, and to function in autonomic control. Furthermore, activity in the orbitofrontal cortex and anterior cingulated cortex have been associated with both sympathetic and parasympathetic effect. Thus, it is believed that stellate ganglion block as described above may be used to treat such psychiatric and behavioral disorders as ADHD and gambling addiction, and to provide relief from the associated compulsive behavior and other symptoms.

The use of the stellate ganglion block to treat mood disorders and psychiatric disorders, including post partum depression and schizophrenia, may be particularly effective in combination with melatonin. Melatonin is a hormone produced by the pineal gland and is involved in synchronizing the body's hormone secretions and setting the brain's internal clock. Typically, the body's production of melatonin begins around dusk, increases during the night, and then decreases as daylight appears. Melatonin supplements are widely used to relieve sleep disorders, including insomnia and the symptoms of jet lag. Melatonin is also used to treat mood disorders and depression, illustratively seasonal affective disorder.

In another embodiment of the present invention, the stellate ganglion block may be used along with melatonin to treat sleep disorders, mood disorders, and depression. In one illustrative embodiment, the stellate ganglion block is performed contemporaneously with administration of melatonin. The melatonin may be administered prior to or subsequent to the stellate ganglion block, illustratively within an hour of the stellate ganglion block, but it is understood that other time periods between the administration of melatonin and stellate ganglion block are within the scope of this invention. Melatonin is often administered orally, but other dosage forms are known and may be used within the scope of this invention. Illustratively, this treatment is performed in the morning, when melatonin levels are lowest. However, this treatment may be effective at other times of the day.

### EXAMPLE 1

Six women suffering from severe menopausal hot flashes each received a standard stellate ganglion block (0.375% Marcaine, 5 cc), as described above. Hot flashes were assessed by self-reporting pre and post stellate ganglion block.

The initial stellate ganglion block was successful in all six subjects, as evidenced by a positive Horner's syndrome and anhydrosis. Successful stellate ganglion block resulted in complete alleviation of hot flashes for times ranging from 2-5 weeks. Patients returned for a follow-up stellate ganglion block after mild hot flashes returned. A second stellate ganglion block produced additional asymptomatic periods of relief ranging from 4-18 weeks. In each case, repeated blocks provided hot flash relief equal to or greater than that of the initial block. Two patients who submitted for a third stellate ganglion block reported 15 and 48 weeks relief.

Accordingly, stellate ganglion blocks appear to be related to relief of hot flashes. Repeat stellate ganglion block results in efficacious multiple week relief of severe hot flashes associated with menopause.

### EXAMPLE 2

A female patient with a ten year history of smoking at least one pack of cigarettes per day received a standard C-6 stellate ganglion block as described in Example 1, for treatment of chronic pain caused by Reflex Sympathy Dystrophy (RSD). Approximately 24-48 hours after receiving treatment, the patient experienced a cessation of the urge to smoke that lasted for a period of about four months, during which the patient stopped smoking completely. At the end of the four month period the urge to smoke returned and the patient received Low Level Laser Therapy for smoking (also referred to as Cold Laser Therapy), which had no effect.

Approximately five months after the initial stellate ganglion block, the patient received a second stellate ganglion block for treatment of RSD. Immediately following the second stellate ganglion block, the patient experienced an immediate 60% to 70% reduction in the desire to smoke, and within 24 hours had no desire to smoke.

Although the invention has been described in detail with reference to preferred embodiments, variations and modifications exist within the scope and spirit of the invention as described and defined in the following claims.

1. A method for treating a patient experiencing the urge to smoke, comprising the step of:
administering a stellate ganglion block to the patient to provide relief from the urge to smoke.

2. The method of claim 1, wherein the stellate ganglion block is administered by delivering an anesthetic to the patient's stellate ganglion.

3. The method of claim 2, wherein the anesthetic is selected from the group consisting of bupivacaine, narcan and combinations thereof.

4. The method of claim 1, wherein the stellate ganglion block is administered by a method selected from the group consisting of: radiofrequency lesioning, linear polarized light irradiation, and surgical sympathectomy of the patient's stellate ganglion.

5. The method of claim 1, wherein the relief is temporary and further comprising the step of administering at least one subsequent stellate ganglion block after recurrence of the urge to smoke.

6. The method of claim 5, wherein the initial stellate ganglion block is administered by delivery of an anesthetic to the patient's stellate ganglion and the subsequent stellate ganglion block is administered by a method selected from the group consisting of: radiofrequency lesioning, linear polarized light irradiation, and surgical sympathectomy of the patient's stellate ganglion.

7. A method for treating a patient experiencing the urge to smoke, comprising the steps of:
administering a plurality of stellate ganglion blocks to the patient over time to provide relief from the urge to smoke, the stellate ganglion blocks administered by delivery of an anesthetic to the stellate ganglion.

8. The method of claim 7, wherein the stellate ganglion blocks are administered on a regular periodic basis.

9. The method of claim 7, wherein the length of time between consecutive stellate ganglion blocks is increasing over time.

10. The method of claim 7, wherein the length of time between consecutive stellate ganglion blocks is at least two weeks.

11. The method of claim 7, wherein the relief from the urge to smoke is temporary and the stellate ganglion blocks are administered after each recurrence of the urge to smoke.

12. The method of claim 7, wherein the anesthetic is bupivacaine.

13. A method for treating a patient having a psychiatric or behavioral disorder, comprising the step of:
administering a stellate ganglion block to the patient to provide relief from the symptoms of the psychiatric or behavioral disorder.

14. The method of claim 13, wherein the stellate ganglion block is administered by delivering an anesthetic to the patient's stellate ganglion.

15. The method of claim 14, wherein the anesthetic is selected from the group consisting of: bupivacaine, narcan and combinations thereof.

16. The method of claim 13, wherein the stellate ganglion block is administered by a method selected from the group consisting of: radiofrequency lesioning, linear polarized light irradiation, and surgical sympathectomy of the patient's stellate ganglion.

17. The method of claim 13, wherein the relief is temporary and further comprising the step of administering at least one subsequent stellate ganglion block after recurrence of the symptoms of post traumatic stress disorder.

18. The method of claim 17, wherein the initial stellate ganglion block is administered by delivery of an anesthetic to the patient's stellate ganglion and the subsequent stellate ganglion block is administered by a method selected from the group consisting of: radiofrequency lesioning, linear polarized light irradiation, and surgical sympathectomy of the patient's stellate ganglion.

19. The method of claim 13, wherein the psychiatric or behavioral disorder is post traumatic stress disorder.

21. The method of claim 13, wherein the psychiatric or behavioral disorder is attention deficit hyperactivity disorder.

19. The method of claim 13, wherein the psychiatric or behavioral disorder is pathological gambling.
